(19) United States
(12) Patent Application Publication
(10) Pub. No.: US 2007/0298991 A1
(43) Pub. Date: Dec. 27, 2007

(54) DENTURE CLEANSER COMPOSITION

(75) Inventor: James J. Longo JR., Wilmington, DE (US)

Correspondence Address: rudoler & derosa llc;ATTN: DOCKET CLERK, 2 BALA PLAZA,, SUITE 300, BALA CYNWYD, PA, 19004, US

(73) Assignee: PREMIER DENTAL PRODUCTS COMPANY, Plymouth Meeting, PA (US)

(21) Appl. No.: 11/763,069

(22) Filed: Jun. 14, 2007

Related U.S. Application Data
(60) Provisional application No. 60/813,987, filed on Jun. 14, 2006.

(51) Int. Cl. A61K 8/00

(52) U.S. Cl. 510/116

(57) ABSTRACT

A solid denture cleaning composition that when dissolved into water produces an acidic solution for the dissolution and subsequent removal of dental deposits such as tartar and calculus present on contaminated dental prosthesis. The preferred composition contains an effective bleaching agent achieved by the incorporation of a hydrogen peroxide/Plasdone linear vinyl pyrrolidone homopolymer. The resulting formula provides a mild bleaching composition, devoid of per-acid salts, per-acids and chlorinating compounds. The formulation is an effective denture cleaner for stubborn stains and contaminants.

### CROSS-REFERENCE TO RELATED APPLICATIONS

The present application claims the benefit of U.S. Provisional Application No. 60/813,987 filed on Jun. 14, 2006, which is incorporated herein by reference.

### STATEMENT REGRADING FEDERALLY SPONSORED RESEARCH DEVELOPMENT

The present invention was not developed with the use of any Federal Funds, but was developed independently by the inventor.

### BACKGROUND OF THE INVENTION

Citric acid has long been known to function as a sequestering agent for heavy metal ions, such as calcium and magnesium. Sulfamic Acid is known for its ability to breakdown and dissolve hard water scale, which primarily occurs in the presence of calcium ions. Calcium and other minerals are primary constituents of tartar and calculus deposits that are formed daily. Mineral ions in conjunction with organic debris form a sticky matrix termed plaque. Plaque is the result of everyday metabolic processes that combine mineral ions such as calcium and phosphate with saliva, partially digested food particles and bacterial cells into a sticky matrix that adheres tenaciously to dental prosthesis.

Over time (as little as 24 hours) this sticky matrix begins to solidify and harden. If not removed promptly, this solidification of soft plaque results in a hardened tartar that eventually leads to calculus. Both tartar and calculus can be very difficult to remove. An effective denture cleaner combines elements that enable the breakdown and dissolution of hardened mineral deposits with elements responsible for the breakdown and elimination of organic biofilm.

thus, there exists the need for a solid denture cleaning composition that when dissolved into water produces an acidic solution for the dissolution and subsequent removal of dental deposits such as tartar and calculus present on contaminated dental prosthesis.

### BRIEF SUMMARY OF THE INVENTION

The invention relates to a denture cleaning composition for dissolution in water forming a resulting solution. In one form of the invention the composition comprises a blend of calcium chelating agents comprising citric acid, sulfamic acid and sodium gluconatea and a bleaching agent. The resulting solution having an acidic pH in the range of about 1.0 to about 3.0.

In another form of the bleaching agent comprises hydrogen peroxide and a film forming polymer for the adherence prevention of calcium based deposits as well as stains. The hydrogen peroxide and film forming polymer may be formed within a single functional complex. The hydrogen peroxide and film forming polymer may also be a hydrogen-bonded complex of hydrogen peroxide and a Plasdone linear vinyl pyrrolidone homopolymer. The hydrogen peroxide/Plasdone complex may contain about 15% to about 25% available hydrogen peroxide, preferably between about 20% available hydrogen peroxide. The peroxide/Plasdone complex may be present within the composition from about 10 to about 30%, preferably between about 15% to about 20%. The Plasdone portion of the hydrogen peroxide/Plasdone complex may have a molecular weight from about 58,000 to about 1,300,000.

In another form of the invention, the bleaching agent is devoid of per-acid salts, per-acids, and chlorinated compounds. The per-acid salts may comprise perborates, persulfates, and percarbonates; the per-acids may comprise peracetic acid, performic acid; and the chlorinated compounds may comprise sodium, calcium, lithium hypochlorites, chlorinated trisodium phosphate, and chlorinated isocyanurates.

The composition may comprise anhydrous citric acid and sulfamic acid in a ratio from about 5:1 to about 10:1, preferably in a ratio of about 8.25:1. The composition may also comprise sodium gluconate from about 0.1 to about 1.0% of the total composition. The composition may further comprise a surface tension reducing agent is selected from a group consisting of anionic, non-ionic cationic and amphoteric surfactants. Preferably, the surface tension reducing agent is sodium dodecylbenzenesulfonate comprising from about 0.1 to about 1.0% of the total composition. The composition may also comprise at least one corrosion inhibitor, such as sodium benzoate comprising from about 0.1 to about 1.0% of the total composition. The composition may further comprise flavoring, fragrance, dyes and an effervescing agent.

### DETAILED DESCRIPTION OF THE INVENTION

The present invention is directed to a denture cleaning composition comprising a mixture of components, that when dissolved in water, result in an acidic solution, highly effective at dissolving and removing contaminating compounds, such as tartar, calculus, stains and biofilms from prosthetic dental appliances.

In the preferred denture cleaning composition, a solid denture cleanser is generated by combining citric acid and sulfamic acid to form an acidic mixture that when hydrated with water, will generate a solution with a pH of about 1.5 to 2.0, but generally not greater than about 3.0. Both citric and sulfamic acid are solid crystalline powder or granular in form and are well suited for the present invention.

The denture cleaning composition also comprises a hydrogen peroxide bleaching agent which yields a composition that provides the ability to effectively dissolve tartar and calculus with the added benefit of a mild bleaching action to remove intrinsic stains that result from different food sources such as juices, coffees and tea as well as biofilms resulting from the growth of bacterial cells. This combination of ingredients provides an acidic mixture with bleaching action, eliminating the need for per-acid salts, such as persulfates, perborates and percarbonates commonly found in existing denture cleaning formulations.

The hydrogen peroxide bleaching agent is preferably in the form of a hydrogen bonded hydrogen peroxide/Plasdone complex that combines hydrogen peroxide and a linear, vinyl pyrrolidone homopolymer into a stable, solid, powdered form, yielding approximately about 20% available hydrogen peroxide in the complex. This complex is sold commercially under the trade name PEROXYDONE, by International Specialty Polymers (ISP). The hydrogen peroxide/Plasdone complex can vary in the molecular weight of the pyrrolidone homopolymer. Typically, the homopolymer can range in molecular weight from about 58,000 to 1,300,000. PEROXYDONE K-30 is the trade name corresponding to the complex containing a homopolymer with an average molecular weight of about 58,000 while PEROXYDONE K-90 corresponds to an average molecular weight of approximately 1,300,000. The greater the molecular weight, the higher the viscosity of the end solution.

In addition to the complex's ability to release hydrogen peroxide into solution, the dissolution of the homopolymer enables the denture cleaning solution to yield film forming properties. Thus, this complex enables the creation of a solid denture cleaner composition with bleaching action and film forming abilities. The greater the molecular weight of the complex, the greater the film forming abilities obtained.

The film forming capabilities of the complex are important in that the denture cleaner formulation can utilize its acid and bleaching chemistries to effectively descale, clean and whiten the prosthetic appliance, while allowing its film forming abilities to leave a residual polymer coating, post cleaning, to aid in future stain and biofilm prevention. Film forming ability of the end solution can be tailored by different combinations of PEROXYDONE K-30 and K-90.

In the preferred composition it has been found that the best results are obtained when the ratio of citric acid to sulfamic acid exists in a ratio from about 5:1 to 10:1, with the greatest results obtained from a ratio of about 8.5:1. Additionally, it has been found that the greatest percentage of hydrogen peroxide/Plasdone complex is about 15-20% of the total denture cleaner formulation. Ideally, the preferred composition will contain approximately 17-18% of the complex within the total formula. At this level, the solid denture cleaner formulation will contain approximately 3.5% available hydrogen peroxide for bleaching activity.

A typical dosage of powder to water in order to generate the aqueous denture cleaning solution would be about 2-3 grams of powder to about 6 fluid ounces (approximately 177 mL) of water. At this ratio, the expected level of hydrogen peroxide in the acid solution would be about 0.03% to 0.06% or from about 300 to about 600 parts per million based on a hydrogen peroxide content of about 20% in the hydrogen peroxide/Plasdone complex. At this level, a sufficient amount of hydrogen peroxide exists in solution to provide effective biofilm removal and bleaching action for stain removal for the appliance.

The preferred composition also relies on the incorporation of an additional chelating ingredient in the form of sodium gluconate. The incorporation of this ingredient functions to add additional sequestration of hard water minerals that may be present on the appliance as well as in the water used to hydrate the powder. Sodium gluconate is the sodium salt of gluconic acid. It is a granular or powdered crystalline solid that is has excellent chelating ability. While it exhibits greater chelating power in alkaline systems, it functions well in the present composition in providing additional chelating ability while providing a mild buffering action for the acid solution, without sacrificing efficacy of the acid chemistries.

In addition, a surface tension reducing agent, a corrosion inhibitor, and antimicrobial agent may also be included in the denture cleaning composition. These ingredients serve important functions by way of allowing the other ingredients within the formula to function efficaciously, without possible compromise to the formulation and/or the prosthetic appliance.

An effective surface tension reducing agent functions to allow the solution to penetrate all of the fissures and cavities that exist on prosthetic dental appliances. Areas between teeth, structural components and gum materials provide a haven for organic and inorganic buildup. It is imperative that the solution be able to penetrate into these restricted areas so that the contamination can be removed. Surfactants provide this needed function by reducing the surface tension of the liquid solution allowing it to penetrate. They also aid in the dissolution of some oleophilic contaminants, such as fats and oils that may be present within the dental contamination as well as the ability to lift the soil from the substrate for further breakdown by the acid and bleaching chemistries.

Suitable surface tension reducing agents are selected from a group comprising of anionic, non-ionic, cationic and/or amphoteric surfactants. The preferred composition utilizes an anionic surfactant, sodium dodecylbenzenesulfonate. Sodium dodecylbenzenesulfonate is sold under the trade name NACCONOL by Stepan Chemical Company. The grade NACCONOL 90G is the preferred form of the surfactant because of its friable, solid flake form that is well suited for a solid denture cleaning composition of the present invention. Other suitable surfactants may comprise, but are not limited to additional anionics, such as sodium lauryl sulfate, sodium lauroyl sarcosinate, sodium lauryl ether sulfate and the like; non-ionics such as alcohol ethoxylates, nonylphenol ethoxylates, and fatty alkyl ethoxylates; cationics such as stearalkonium chloride, didecyldimethyl ammonium chloride and the like; and amphoterics such as cocoamidopropylamine oxide, laurylamidopropyl betaine and the like. Other surfactants that function in the presence of acids would be suitable as well.

The preferred composition additionally comprises an antimicrobial agent and corrosion inhibitor. Sodium benzoate converts to benzoic acid in the presence of acid mixtures. Benzoic acid is an effective antimicrobial agent for the control of bacteria, yeasts and mold, all potential contaminants of removable prosthetics. Because sodium benzoate only converts to benzoic acid in acid environments (pH below approximately 3.5) it is well suited for the present invention as an effective antimicrobial agent. Sodium benzoate is available in a dense powdered or granular form, well suited for the solid denture cleaning formulation of the present invention.

In addition to its antimicrobial properties, sodium benzoate functions well as a corrosion inhibitor for stainless steel, a primary constituent of removable prosthetic dental appliances. It is also well suited at protecting soldered joints which are an area of great concern for corrosion and discoloration. Generally, care must be employed with acid cleaners because of discoloration to certain metals. Utilization of the sodium benzoate in the formula allows for the formula to employ an acid chemistry (pH below 3.0), which is highly effective at removing tartar and calculus deposits, while remaining safe for the metallic structures of the prosthesis. Additionally, other combinations such as sodium nitrite and the like can add additional corrosion inhibition benefits to the formula when combined with sodium benzoate.

The preferred composition may also contain a fragrance and dye. Fragrances such as mint, work well with the existing formulation. Mint fragrances can be derived from spearmint oils, peppermint oils and the like. Additional fragrance may include fruits, florals, vanilla and the like. The preferred fragrance is peppermint. The preferred dye is an FD&C and/or D&C yellow dye or any mixture thereof.

The denture cleaning composition of the present invention may also contain ingredients that render the formula more esthetically pleasing. Ingredients such as flavoring agents and sweeteners can be employed to aid in the acceptance of the appliance back into the mouth post cleaning.

Additionally, ingredients may be employed that would render the solid denture cleaning composition to be easier to process and use. Ingredients such as flow agents, lubricants and effervescing agents can be utilized as well. Flow agents such as amorphous fumed silica may be employed to scavenge moisture within the formula. This prevents caking of the product. Thus, the powder will remain loose and free-flowing for filling into the appropriate packaging.

The denture cleaner composition of the present invention is primarily used as a powder or granular form; however, the invention could be formed into a tablet or combined with water to form a concentrated solution. The preferred packaging for the preferred composition is in pre-measured unit dose pouches or sachets, supplying enough powdered or granular material to generate one denture cleaning application. If tabletting of the formula were the preferred means of delivery for the composition, ingredients such as magnesium stearate could be added to aid in the lubrication of the formula for release from the tablet press. Additionally, effervescing agents such as sodium bicarbonate, sodium carbonate or mixtures thereof could be employed to the formula.

### EXAMPLES OF THE PREFERRED COMPOSITION

### Example 1

A denture cleaning composition was prepared comprising the following:

| | |
|---|---|
| Citric acid anhydrous | 72.00% |
| Sulfamic acid crystalline | 9.00% |
| PEROXYDONE K-30 | 17.65% |
| Sodium Gluconate | 0.48% |
| Sodium Dodecylbenzenesulfonate (NACCONOL 90G) | 0.41% |
| Sodium Benzoate, dense granular | 0.41% |
| Fresh Peppermint fragrance | 0.04% |
| D&C Yellow #8 powdered dye | 0.01% |

This formula was combined in a planetary mixer by first combining the fragrance with the sodium gluconate and the dye with the sodium benzoate and then adding the citric acid, sulfamic acid, Peroxydone K-30 and Nacconol 90G respectively. This formula was then added to water in a ratio of 2.0 grams of denture cleaner to 177.0 grams of warm tap water. The solution was stirred briefly to dissolve all materials and then a pH was taken. The pH for the denture cleaning solution was 1.8+/−0.20. The solution was then titrated for available hydrogen peroxide and produced a reading of 0.0374% available hydrogen peroxide or 374 parts per million.

The resulting solution was then challenged against an in vitro tartar composition comprising calcium sulfate, calcium acetate, sodium phosphate, human saliva, and sucrose solution. The resulting mixture was allowed to remain moist in an incubator at 40 degrees Celsius for 24 hours to support microbial growth within the mixture. The sticky mixture was then applied liberally to three removable prosthetic appliances taking care to coat hard to access areas between the teeth and wire support structures. The sticky matrix was then dried and heat fixed onto the prosthetic dental appliances by utilizing a forced, hot air pyro-heater. Once dry, the appliances were allowed to cool and set for 24 hours at room temperature.

The appliances were then placed into 175 mL of the acid denture cleaning solution of the present invention, a solution of Efferdent® and a control of tap water. All solutions were at room temperature. The three solutions were allowed to soak statically with observations taken at 15 minutes, 30 minutes and one hour. The results were tabulated.

Results:

15 Minutes

After 15 minutes, the sticky matrix was dissolving rapidly with close to 50% of the tartar mixture removed from the appliance in the acid cleaner solution of the present invention.

The Efferdent® solution removed slightly less than the acid cleaner, mainly as a result of the effervescent action dislodging the debris.

The tap water control had removed little to no material.

30 Minutes

Only a small portion, approximately less than 10%, of the debris remained on the prosthetic appliance in the acid solution of the present invention, with no traces of the removed material within the beaker.

The Efferdent® solution still had a significant amount of tartar debris affixed within the spaces between the teeth. There were also pieces of the dislodged debris present on the bottom of the beaker, intact and undissolved.

The tap water had only a slight removal of less than approximately 10% of the debris.

1 Hour

After one hour, all of the tartar debris had been removed from the dental appliance soaked in the acid solution of the present invention, with no traces of the dislodged debris on the bottom of the beaker.

The Efferdent® solution still had an amount of tartar debris affixed within the spaces between the teeth. The solution had changed color from a dark blue to a light blue indicating a bleaching effect due to the release of oxygen gas. There were still pieces of the dislodged debris present on the bottom of the beaker, intact and undissolved.

The tap water control still had a significant amount of debris on the appliance.

Corrosion Test

Additionally, the prosthetic appliance was allowed to soak for 24 hours in the acid solution of the present invention with no discoloration or corrosion to the metal clasps and solder joints as well as no damage to the acrylic structures and teeth.

### CONCLUSION

The results show the ability of the present invention to remove and dissolve tartar and calculus present on dental appliances in a brief static soak without compromising the integrity of the appliance itself. The resulting formula provides a solution with greater ability to clean difficult stains and debris.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A solid denture cleaning composition for dissolution in water forming a resulting solution, comprising:
a blend of calcium chelating agents comprising citric acid, sulfamic acid and sodium gluconate; and
a bleaching agent, wherein the resulting solution having an acidic pH in the range of about 1.0 to about 3.0.

2. The solid denture cleaning composition according to claim 1 wherein the bleaching agent comprises hydrogen peroxide and a film forming polymer for the adherence prevention of calcium based deposits as well as stains.

3. The solid denture cleaning composition according to claim 2 wherein the hydrogen peroxide and film forming polymer are formed within a single functional complex.

4. The solid denture cleaning composition according to claim 2 wherein the hydrogen peroxide and film forming polymer are a hydrogen-bonded complex of hydrogen peroxide and a Plasdone linear vinyl pyrrolidone homopolymer.

5. The solid denture cleaning composition according to claim 4 wherein the hydrogen peroxide/Plasdone complex contains about 15% to about 25% available hydrogen peroxide.

6. The solid denture cleaning composition according to claim 4 wherein the hydrogen peroxide/Plasdone complex contains about 20% available hydrogen peroxide.

7. The solid denture cleaning composition according to claim 4 wherein the hydrogen peroxide/Plasdone complex is present within the composition from about 10 to about 30%.

8. The solid denture cleaning composition according to claim 4 wherein the hydrogen peroxide/Plasdone complex is present within the composition from about 15% to about 20%.

9. The solid denture cleaning composition according to claim 4 wherein the Plasdone portion of the hydrogen peroxide/Plasdone complex has a molecular weight from about 58,000 to about 1,300,000.

10. The solid denture cleaning composition according to claim 1 wherein the bleaching agent is devoid of per-acid salts, per-acids, and chlorinated compounds.

11. The solid denture cleaning composition according to claim 10 wherein the per-acid salts comprise perborates, persulfates, and percarbonates; the per-acids comprise peracetic acid, performic acid; and the chlorinated compounds comprise sodium, calcium, lithium hypochlorites, chlorinated trisodium phosphate, and chlorinated isocyanurates.

12. The solid denture cleaning composition according to claim 1 further comprising anhydrous citric acid and sulfamic acid in a ratio from about 5:1 to about 10:1.

13. The solid denture cleaning composition according to claim 1 further comprising anhydrous citric acid and sulfamic acid in a ratio of about 8.25:1.

14. The solid denture cleaning composition according to claim 1, further comprising sodium gluconate from about 0.1% to about 1.0% of the total composition.

15. The solid denture cleaning composition according to claim 1, further comprising a surface tension reducing agent.

16. The solid denture cleaning composition according to claim 15 wherein the surface tension reducing agent is selected from a group consisting of anionic, non-ionic cationic and amphoteric surfactants.

17. The solid denture cleaning composition according to claim 15 wherein the surface tension reducing agent is sodium dodecylbenzenesulfonate comprising from about 0.1% to about 1.0% of the total composition.

18. The solid denture cleaning composition according to claim 1 further comprising at least one corrosion inhibitor.

19. The solid denture cleaning composition according to claim 15 wherein the corrosion inhibitor is sodium benzoate comprising from about 0.1 to about 1.0% of the total composition.

20. The solid denture cleaning composition according to claim 1 further comprising flavoring, fragrance, dyes and an effervescing agent.

21. A denture cleaning composition comprising:
anhydrous citric acid from about 70 to about 85%;
sulfamic acid from about about 5 to about 15%
a hydrogen peroxide/Plasdone complex from about 15 to about 20%;
sodium gluconate from about 0.1 to about 1%;
sodium dodecylbenzenesulfonate from about 0.1 to about 1%;
sodium benzoate from about 0.1 to about 1%;
a flavor from about 0.05 to about 0.5%;
a fragrance from about 0.05 to about 0.5%; and
a dye from about 0.01 to about 0.1%.

22. The solid denture cleaning composition for dissolution in water forming a resulting solution, comprising:
a blend of calcium chelating agents comprising citric acid, sulfamic acid and sodium gluconate;
a bleaching agent, the bleaching agent comprising hydrogen peroxide and a film forming polymer for the adherence prevention of calcium based deposits as well as stains;
anhydrous citric acid and sulfamic acid in a ratio from about 5:1 to about 10:1;
sodium gluconate from about 0.1 to 1.0% of the total composition;
a surface tension reducing agent is selected from a group consisting of anionic, non-ionic cationic and amphoteric surfactants; and
a corrosion inhibitor is sodium benzoate comprising from about 0.1 to about 1.0% of the total composition, wherein
the resulting solution having an acidic pH in the range of about 1.0 to about 3.0.

23. The solid denture cleaning composition according to claim 22 wherein the hydrogen peroxide and film forming polymer are formed within a single functional complex.

24. The solid denture cleaning composition according to claim 22 wherein the hydrogen peroxide and film forming polymer are a hydrogen-bonded complex of hydrogen peroxide and a Plasdone linear vinyl pyrrolidone homopolymer.

25. The solid denture cleaning composition according to claim 22 further comprising flavoring, fragrance, dyes and an effervescing agent.
